(19) United States
(12) Patent Application Publication
(10) Pub. No.: US 2003/0113347 A1
(43) Pub. Date: Jun. 19, 2003

(54) Immunostimulating and immunopotentiating reconstituted influenza virosomes and vaccines containing them

(75) Inventors: Maria Grazia Cusi, Siena (IT); Reinhard Gluck, Bern-Spiegel (CH); Ernst Walti, Munchenbuchsee (CH)

Correspondence Address: Pillsbury Winthrop LLP, Intellectual Property Group, Suite 200, 11682 EI Camino Real, San Diego, CA, 92130, US

(73) Assignee: SCHWEIZ. SERUM- & IMPFINSTITUT BERN

(21) Appl. No.: 10/269,501

(22) Filed: Oct. 10, 2002

Related U.S. Application Data
(63) Continuation of application No. 09/264,551, filed on Mar. 8, 1999, now abandoned.
Application No. 09/264,551 is a continuation-in-part of application No. 08/225,740, filed on Apr. 11, 1994, now Pat. No. 5,879,685.
Application No. 09/264,551 is a continuation-in-part of application No. 07/965,246, filed on Mar. 3, 1993, now Pat. No. 5,565,203.

(30) Foreign Application Priority Data
May 22, 1998 (WO) PCT/EP98/03050
May 8, 1992 (WO) PCT/EP92/01014
May 23, 1997 (EP) EP97108390.2
May 8, 1991 (EP) EP91107527.3
May 10, 1991 (EP) EP91107647.9

(51) Int. Cl. A61K 39/145; A61K 39/29

(52) U.S. Cl. 424/226.1; 424/210.1

(57) ABSTRACT

Described are virosomes comprising cationic lipids, biologically active influenza hemagglutinin protein or biologically active derivatives thereof and nucleic acids encoding antigens from pathogenic sources in their insides, preferably antigens from mumps virus wherein said antigens are derived from conserved external and internal proteins of said virus. Provided are virosomes which may advantageously be formulated as vaccines capable of inducing strong neutralizing antibody and cytotoxic T cell responses as well as protection to pathogenic sources such as a mumps virus. Furthermore, vaccines comprising recombinant DNA derived from DNA encoding conserved external and internal proteins from mumps virus are described.

This application claims the benefit of priority under 35 USC §120 of PCT application serial No. PCT/EP98/03050, filed May 22, 1998, which claims priority from European application serial no. EP97108390.2, filed May 23, 1997, and the present application is a continuation-in-part of U.S. application Ser. No. 08/225,740, filed Apr. 11, 1994 (pending), which is a continuation-in-part application of U.S. application Ser. No. 07/965,246, filed Mar. 3, 1993 (issued), which claims priority from PCT application no. PCT/EP92/01014, filed May 8, 1992, European application No. EP91107527.3 filed May 8, 1991 and European application no. 91107647.9, filed May 10, 1991. The disclosure of the prior applications is considered part of (and is incorporated by reference in) the disclosure of this application.

The present invention relates to virosomes comprising cationic lipids, biologically active influenza hemagglutinin protein or biologically active derivatives thereof and nucleic acids encoding antigens from pathogenic sources in their insides. The nucleic acids are most advantageously DNA. It is preferred that the DNA encodes antigens from mumps virus wherein said antigens are derived from conserved external and internal proteins of said virus. The virosome of the invention may advantageously be formulated as vaccines. It could be show-n in accordance with the present invention that such vaccines induce strong neutralizing antibody as well as cytotoxic T cell responses. Most importantly, protection to pathogenic sources such as a mumps virus could be demonstrated. The present invention further relates to vaccines comprising recombinant DNA derived from DNA encoding conserved external and internal proteins from mumps virus.

The use of purified preparations of plasmid DNA (deoxyribonucleic acid) constitutes a new approach to vaccine development. Plasmid DNA vaccines may find application as preventive vaccines, immunizing agents for the preparation of hyperimmune globuline products or diagnostics and therapeutic vaccines for infectious diseases or for other indications such as cancer. Plasmid DNA vaccines are defined as purified preparations of plasmid DNA designed to contain a gene or genes for the intended vaccine antigen as well as genes incorporated into the construct to allow for production in a suitable host system. Plasmid DNA vaccines currently under development are constructs derived from bacterial plasmids that contain one or more genes from an infectious agent. These plasmids possess DNA sequences necessary for selection and replication in bacteria, eukaryotic promoters and enhancers and transcription termination/polyadenylation addition sequences for gene expression.

In order to avoid the injection of high amounts of DNA for vaccination efficient gene transfer techniques have ro be employed for an acceptable vaccine in humans. The ability to introduce cloned genes into cells, generally referred to as transformation or transfection, is one of the most powerful and far-reacting methodologies to come out of molecular biology. It has played a critical role in the study of gene expression and protein structure and function. However, many standard techniques work on only limited ranges of host cells and others are labor intensive or require large numbers of cells. The advantages and disadvantages of current gene transfer techniques can be summarized as follows:

a) Virus mediated gene transfer: Genes can be introduced stably and efficiently into mammalian cells by retroviral vectors. However, the efficiency is very low for cells that are non-replicating because retroviruses infect only dividing cells. Further, general safety concerns are associated with the use of retroviral vectors relating to, for instance, the possible activation of oncogenes. Replication-defective adenovirus has become the gene transfer vector-of-choice for a majority of investigators. The adenovirus vector mediated gene delivery involves either the insertion of the desired gene into deleted adenovirus particles or the formation of a complex between the DNA to be inserted and the viral coat of adenovirus by a transferrin-polylysine bridge. The drawback of this very efficient system in vivo is an undefined risk of infection or inflammation: Despite the El gene deletion that renders the virus defective for replication, the remaining virus genome contains numerous open reading frames encoding viral proteins (Yang et al. 1994; Proc. Natl. Acad. Sci. USA 91, 4407-4411). Expression of viral proteins by transduced cells elicits both humoral and cellular immune responses in the animal and human body and thus, may provoke inflammation and proliferation.

In the HVJ (Sendai virus) mediated method the foreign DNA is complexed with liposomes. The liposomes are then loaded with inactivated Sendai virus (hemagglutinating virus of Japan; HVJ). This method has successfully been used for gene transfer in vivo to many tissues. In addition, cellular uptake to antisense oligonucleotides by HVJ-liposomes was reported (Morishita et al. 1993; J. Cell. Biochem. 17E, 239). A particular disadvantage is, however, that the HVJ-liposomes show non-specific binding to red blood cells.

b) Lipid mediated gene transfer. Positively charged liposomes made of cationic lipids appear to be safe, easy to use and efficient for in vitro transfer of DNA and antisense oligonucleotides. The highly negatively charged nucleic acids interact spontaneously with cationic liposomes. Already by simple mixing of the polynucleotides with preformed cationic liposomes a complete formation of DNA-liposome complexes is achieved.

However, the in vivo transfection efficiency is very low and the incubation times are long, because there is no cell specific marker on the membranes of cationic liposomes. Further, it cannot be excluded that large amounts of cationic lipids exhibit toxic effects in vivo.

c) Biolistics as gene transfer methods: The term “biolistics” (biological ballistics) is used to define processes that literally shoot high velocity microprojectiles, carrying DNA, into cells. The biolistic process was originally developed by Sanford et al. (Sanford, J. C., Klein, T. M., Wolf, E. D., Allen, N.: Delivery of substances into cells and tissues using a particle bombardment process. J. Part. Sci. Technol. 1987, 5: 27-37) as a means of introducing DNA into plant cells. The limitations of existing, methods of gene transfer stimulated the idea of shooting tungsten or gold particles coated with DNA directly into cells. Since then its use in transfection has extended well beyond plants to an ever-growing list of cell types, some of which had previously been recalcitrant to more routine methods of gene transfer. Several improved particle acceleration devices have been developed and in most current designs gunpowder is replaced by pressurized helium. The only commercially available apparatus is the PDS-1000 (DuPont/Biorad). The main drawback of using gun-like performances for medical applications such as vaccination, however, remains.

Accordingly, the technical problem underlying the present invention was to overcome the disadvantages associated with the development of the prior art nucleic acid vaccines and provide a means that can successfully be used in the formulation of highly protective and safe vaccines.

The solution to said technical problem is achieved by providing the embodiments characterized in the claims. Thus, the present invention relates to a vaccine comprising a virosome, said virosome comprising

(a) a cationic lipid;

(b) an influenza hemagglutinin protein (HA) or a derivative thereof which is biologically active and capable of inducing the fusion of said virosome with cellular membranes and of inducing the lysis of said virosome after endocytosis by antigen presenting cells; and

(c) a nucleic acid comprising a nucleic acid encoding an antigen derived from a pathogen located in the inside.

The vaccine of the invention optionally comprises a pharmaceutically acceptable carrier and/or diluent and is preferably formulated according to conventional protocols.

The term “cationic lipid” as used herein refers to-cationic and/or polycationic lipids. Said term thus describes an organic molecule that contains a cationic component and a nonpolar tail, a so-called head-to-tail amphiphile, such as N-[(1,2,3-dioleoyloxy)propyl]-N, N, N-trimethylammonium chloride (DOTMA)(Felgner et al. 1987; Proc. Natl. Acad. USA 84: 7413-7417), N-[1,2,3-dioleoyloxy)-propyl]-N,N,N-trimethylammonium-methyl-sulfate (DOTAP); N-t-butyl-N′-tetradecyl-3-tetradecylaminopropionamidine (Ruysschaert et al. 1994; Biochem. Biophys. Res. Commun. 203: 1622-1628). The term in particular includes the below defined polycationic lipids. The term “polycationic lipid” refers to an organic molecule that contains a polycationic component and a nonpolar tail such as the lipospermine: 1,3-dipalmitoyl-2-phosphatidylethanolamido-spermine (DPPES) and dioctadecylamidoglycyl-spermine (DOGS) (Behr et al. 1989; Proc. Natl. Acad. USA 86, 6982-6986); 2,3-dioleoyloxy-N-[2(sperminecarboxamido)ethyl]-N, N-dimethyl-1-propane-aminium-trifluoracetate (DOSPA); 1,3-dioleoyloxy-2-(6-carboxy-spermyl)-propylamide (DOSPER); N,N,N′,N′-tetramethyl-N,N′-bis(2-hydroxyethyl)-2, 3-dioleoyloxy-1,4-butanediammonium iodide (THDOB).

The cationic lipids used in accordance with the present invention optionally contain phospholipids such as phosphatidylethanolamine and phosphatidylcholine. It has proved advantageous to choose a lipid composition of the membrane comprising-band on total lipids, either

(i) 90% by weight of cationic lipids, for example comprising polycationic lipids and 10% influenza virus envelope phospholipids; or

(ii) 80 to 90% by weight of cationic lipids, for example comprising polycationic lipids, 5 to 10% influenza virus envelope phospholipids and 5 to 10% by weight of phosphatidyl-ethanolamine; or

(iii) 40 to 80% by weight of cationic lipids, comprising, for example, polycationic lipids, 5 to 20%by weight of influenza virus envelope phospholipids, 5 to 15% by weight of phosphatidyl-ethanolamine and 5 to 50% by weight of phosphatidyl-choline.

The cationic vesicles with the HA component advantageously have a mean diameter of approximately 100-200 nm and a completely closed lipid bilayer. The structure of the cationic bilayer membrane is such that ′the hydrophilic, positively charged beads of the lipids are oriented towards the center of the bilayer.

Unlike known liposomal compositions for delivery of nucleic acids, cationic virosomes need not fuse with or destabilize the plasma cell membrane to enter the cytoplasm. Cationic virosomes enter the host cells via a two step mechanism: (1) attachment and (2) penetration. In the first step they bind via hemagglutinin and/or the cell-specific markers to cell receptors, particularly to membrane glycoproteins or glycolipids with a terminal sialic acid, and are then very efficiently incorporated by receptor-mediated endocytosis.

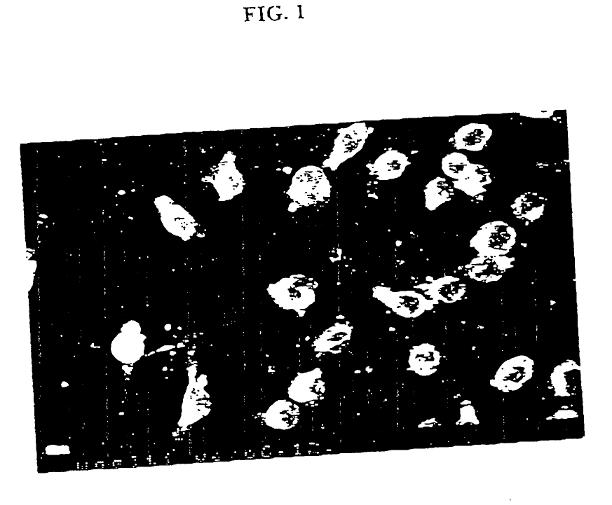

in accordance with the present invention, the term “influenza hemagglutinin protein (HA) or derivative thereof which is biologically active and capable of inducing the fusion of said virosome with cellular membranes and of inducing the lysis of said virosome after endocytosis by antigen presenting cells” relates to (poly)peptides which substantially display the full biological activity of native hemagglutinin and are thus capable of mediating the adsorption of the cationic vesicles of the present invention to their target cells via sialic acid containing receptors. In accordance with the present invention, it could be shown by electron microscopy that the reconstituted viral spike proteins (hemagglutinin and preferably also neuraminidase) are integrated in the lipid bilayer and extend from the surface of the cationic vesicles (FIG. 1). The biologically active hemagglutinin referred to in this specification preferably refers to the fusion peptide which is incorporated into the trimeric hemagglutinin molecule of influenza virus. Also, biologically active hemagglutinin may refer to the complete hemagglutinin trimer of viral surface spikes or to one monomer or to one or both cleaved subunits, the glycopeptides HA1 and HA2, containing the functional fusion peptide. In another embodiment, said term refers to the fusion peptide itself, isolated or synthetically produced. Thus, the fusion peptide mediates the entry of the plasmid-influenza envelope complex into the cytoplasm by a membrane-fusion event and finally leads to the release of the transported plasmid into the cell where it will be expressed. It is envisaged that the virosomes are incorporated via receptor-mediated endocytosis in the course of which the virosomes get entrapped in endosomes. The developing acidic pH (pH 5-6) within the endosomes activates the hemagglutinin fusion peptide and triggers the fusion of the virosomal membrane with the endosomal membrane (Wiley, D. C. and Skehel, J. J., Ann. Rev. Biochcm. 56 (1987), 365). The membrane fusion reaction opens the lipid envelope of the virosomes and liberates the entrapped genetic material into the cytosol. Thus, due to the hemagglutinin portion, preferably the functionally active fusion peptides of the present virosomes the encapsulated material is released shortly after endocytosis so as to avoid an undesired long stay in the endosomes which would give rise to unspecific degradation of the substances contained in the virosomes. The molecules mechanisms underlying the subsequent expression of said genetic material is expected to follow conventional and well-known rules.

The reconstituted virosomes of the present invention have essentially the same fusion activity towards target cells as the intact virus from which they are reconstituted. Preferably, the comparison of fusogenicity is drawn to intact influenza A virus. The fusion activity is measured according to known procedures, preferably as reported by Hoekstra et al. (1984), Biochemistry 23: 5675-5681 and Luscher et al. (1993), Arch. Virol. 130: 317-326. In order to achieve the best possible results it proved advantageous to first carefully isolate and purify the hemagglutinin glycoproteins. In this way, there is no inactivation either by proteolytic digestion or by reduction of its intramolecular S—S bonds.

The HA or derivative thereof may be obtained from natural sources, it may further be of recombinant or semisynthetic origin or maybe chemically produced.

The vaccine of the present invention has the additional advantage that large DNA concentrations in the vaccine are avoided.

The term ‘nucleic acid comprising a nucleic acid encoding an antigen derived from a pathogen” refers to nucleic acids carrying, for example, mumps genes or other microbial genes. Said nucleic acids encode at least one antigen from a pathogenic source. Advantageously, said nucleic acids are cloned under appropriate promoter control. The corresponding construct is a vector and preferably a plasmid. The preferred inoculated plasmid DNA seems to persist episomally without replication in the nuclei of myocytes without integrating into the genome. Expression of antigens after intramuscular plasmid DNA injection has been shown in striated muscle cells (Felgner Ph. L., Tsai. Y. J., Felgner J. H.: Advances in the design and application of cytofectin formulations. Chapter 4. In: Handbook of Nonmedical Applications of Liposomes. Vol. IV, Editors: D. D. Lasic, Y. Barenholz, CRC Press, Boca Raton, New York, London, Tokyo, 1996). This may cause persistent antigen presentation leading to prolonged stimulation of the immune response. DNA-based vaccination efficiently induces MHC class I-restricted cytotoxic T lymphocyte (CTL) responses and serum antibody responses to different antigens.

The term “antigen” as used herein denotes a two-or three-dimensional proteinaceous, including lipoproteinaceous and glycoproteinaceous structure forming at least one epitope specific for a pathogen that is recognized in a B cell or T cell response. The antigen is “derived” from the pathogen e.g. by using a nucleic acid directly obtained from said pathogen which is then translated. The term “derived” also includes that the nucleic acid encoding said antigen which was obtained from a natural source has been altered by recombinant means, as long as the immunological characteristics leading to protection against the pathogenic features of said source remain essentially unaltered. Said nucleic acids as well as the antigens may also be produced by synthetic or semisynthetic methods.

Preferably, the virosome of the invention also comprises intact neuraminidase molecules that are preferably also derived from influenza virus. Viral neuraminidase (NA) is an exoglycosidase that hydrolyzes terminal sialic acid residues from any glycoconjugate, including the viral glycoprotein themselves. The virion NA spikes are tetramers of the NA molecules that are anchored in the lipid bilayer by an amino-terminal hydrophobic amino acid sequence (Shaw, M. W., et al., 1992: New Aspects of Influenza Viruses. Clin. Microbial. Reviews, 74-92). Recently it could be demonstrated that inhibition of the neuraminidase activity, e.g. through antibodies, leads to the reduction of influenza infectivity in human.

In a preferred embodiment of the vaccine of the invention, said cationic lipid is an organic molecule that contains a (poly)cationic component and a non-polar tail, wherein said (poly)cationic compounds comprise at least one member selected from the group consisting of:

N-[1,2,3-dioleoyloxy)propyl]-N,N,N-trimethylammonium chloride (DOTMA)

N-[1,2,3-dioleoyloxy)propyl]-N,N,N-trimethylammoniummethylsulfate (DOTAP)

N-t-butyl-N′-tetradecyl-3-tetradecylaminopropionamidine; and

the polycationic lipids comprise at least one member selected from the group consisting of 1,3-dipalmitoyl-Z-phosphatidylethanolamido-spermine (DPPES), diootadecylamidoglycyl spermine (DOGS), 2,3-dioleyloxy-N-[sperminecarboxamido)ethyl)-N,N-dimethyl-1-propane-aminiumtrifluoroacetate (DOSPA), 1,3-dioleoyloxy-2-(6-carboxy-spermyl)-propylamide (DOSPER) and N,N,N′,N′-tetramethyl-N,N′-bis(2-hydroxyethyl)-2,3-dioleoyloxy-1,4-butanediammoniurn iodide (THDOB).

It could be shown in accordance with the present invention that the above recited components have particular and surprising properties. When these substances are mixed together in an aqueous environment, the two macromolecular systems associate ionically and the lipids and DNA reorganize in close association with each other.

In a further preferred embodiment of the vaccine of the present invention, said nucleic acid is DNA. In this embodiment, the nucleic acid is advantageously cloned in DNA vectors which are particularly stable in comparison to RNA molecules.

In another preferred embodiment of the invention, said nucleic acid is RNA. This embodiment may be advantageous if direct expression of the nucleic acid is desired, i.e. the nucleic acid has not to enter the nucleus to be transcribed into expressible RNA.

In accordance with the present invention, it is additionally preferred that the nucleic acid contained in said virosome is a polycistronic acid.

The various cistrons may encode at least two antigens of the same or different pathogens. For example, one cistron may encode an antigen of mumps virus and the other cistron may encode an antigen from a different microbial source.

Coexpression of different proteins in stoichiometrically defined ratios within a single cell can be achieved by polycistronic expression constructs. Following the intramuscular inoculation of “naked” plasmid DNA encoding an antigen, humoral and cellular immune response against the respective antigen expressed by the construct can be primed. The use of polycistronic nucleotide vectors in DNA-based immunization allows the use of at least two novel options for genetic immunizations:

(1) Polycistronic nucleotide vectors can be used to deliver with a single injection a multivalent vaccine that efficiently stimulates a broad spectrum of immune reactivities against several antigens from the same or different pathogens.

(2) Polycistronic vectors can be constructed that limit the life span of the in vivo transfected cell. This is achieved by co-expressing an inducible suicide gene within the antigen-presenting cell. The construct thereby allows expression of the antigen for a few weeks, sufficient to prime an immune response, but allows subsequent elimination of cells expressing the foreign expression constructs.

Accordingly, a particularly preferred embodiment according to the invention concerns a polycistronic construct, which is characterized by a suicide gene preferably inducible with a therapeutically acceptable drug.

The suicide gene may be comprised in the nucleic acid together with one or more nucleic acid sequences encoding antigenes from the same or different pathogens.

In an additional preferred embodiment of the vaccine of the invention, said pathogen is a bacterium, a prion, a parasite or a virus.

It is particularly preferred that said virus is a single-stranded, non-segmented, genome negative-sense RNA virus, preferably of the family Paramyxoviridae and most preferably mumps virus or measles virus.

The mumps virus belongs to the paramyxoviridae, subclass paramyxovirus. It is a pathogen causing the contagious infantile illness which consists of the inflammation of parotid glands. During the incubation period following infection, the virus replicates in the respiratory epithelium and then disseminates into secretory ducts of the parotid glands. Other glands may become infected thereafter and numerous cases of meningitis have been reported. Among complications related to the infection, encephalitis is a serious one with a mortality rate of about 1%; deafness cases have also been reported.

A vaccine against mumps is available: it is made of an attenuated live virus, produced by culturing infected embryonic chicken cells or human diploid cells. The vaccine leads to the seroconversion in vaccinated individuals in about 90-95% but the protection rate in the field is far smaller than expected from the seroconversion rate. In addition several “classical” mumps vaccine strains had to be withdrawn from the market due to a high encephalotropic potential after vaccination. Furthermore, it is known that live mumps virus vaccines are relatively low in heat stability reducing their use in the field, specially in developing countries, where it is difficult to maintain a cold chain.

As has already been stated herein above, it is preferred that said nucleic acid is a recombinant vector, preferably a plasmid.

It is particularly preferred that the “naked” mumps DNA plasmids contain genes encoding the hemagglutinin-neuraminidase (HN) antigen of mumps virus, the fusion (F) protein of mumps virus and the nucleoprotein (NP) of mumps virus. These plasmids also possess DNA sequences necessary for selection and replication in bacteria, eukaryotic promoters and enhancers and transcription termination/polyadenylation addition sequences for gene expression.

Accordingly, in a particularly preferred embodiment, the invention provides an influenza enveloped mumps DNA vaccine which contains the following components:

(a) a mumps virus-derived polynucleotide which induces protective immune response upon introduction into vertebrate tissue,

(b) a mixture of phospholipids including influenza envelope phospholipids and cationic and/or poly-cationic lipids,

(C) a mixture of biologically active influenza glycoproteins containing fusogenic hemagglutinin and intact neuraminidase.

Construction of mumps polynucleotide monocistronic expression vectors or polycistronic expression vectors may be done as follows:

(1) pCMV promoter insertion and construction of mono-or poly-cistronic expression vectors: Promoter sequence of the immediate early region of the human cytomegalovirus or of the desmin gene have been shown to support expression of an immunogenic gene product after intramuscular injection of plasmid DNA. Recombinant plasmids of this invention contain one or several gene inserts of mumps virus or other microbial agents (e.g. hepatitis A, B, C, D or E-virus, RSV, Dengue virus, HIV, Rabies virus, Influenza virus, Measles virus, Parainfluenza virus, Rhinovirus, Pseudomonas, Klebsiella, Escherichia coli, Salmonella typhi, Haemophilus influenzae, Bordetella pertussis or Plasmodium falciparum). The fusion between two vectors can generate dicistronic PCMV, etc.

(2) Polycistronic expression constructs that express most proteins from mumps virus, other microbial agent or a chimeric construct from different microbial agents (e.g. measles, mumps and rubella): E. g. mumps NP, HN and F.

This most preferred expression construct according to the invention may also be characterized in that the CMVp sequence is replaced for the SV40p sequence.

As has also been stated herein above, it is particularly preferred to employ the HA fusion peptide as the HA derivative.

The invention further relates to a vaccine comprising a vector encoding the hemagglutinin-neuraminidase antigen of mumps virus, the fusion protein of mumps virus and the nucleoprotein of mumps virus.

It is particularly preferred that said vector is GC9, GC23 or GCNP or GCDC described in the examples hereinafter.

In another embodiment, the present invention relates to a method simulating the immune system of a patient in need thereof, comprising administering a suitable dosage of the vaccine described herein above. For example, a suitable dosage may be in the range of

| | |
|---|---|
| Influenza HA | 1-50 mcg |
| Total Phospholipid | 50 mcg-10 mcg |
| Plasmid | 0.1 mcg-100 mcg |

In another embodiment, the aforedescribed method is for the prophylaxis of infectious diseases.

In a preferred embodiment of the vaccines and methods of the present invention the above described vaccines are designed to be administered via nasal routes.

The design and formulation, respectively, may be effected according to conventional procedures.

The figures show:

FIG. 1: Immunofluorescence test carried out on Vero cells infected by DOTAP-virosomes encapsulating mumps plasmids by using anti-mumps polyclonal antibodies

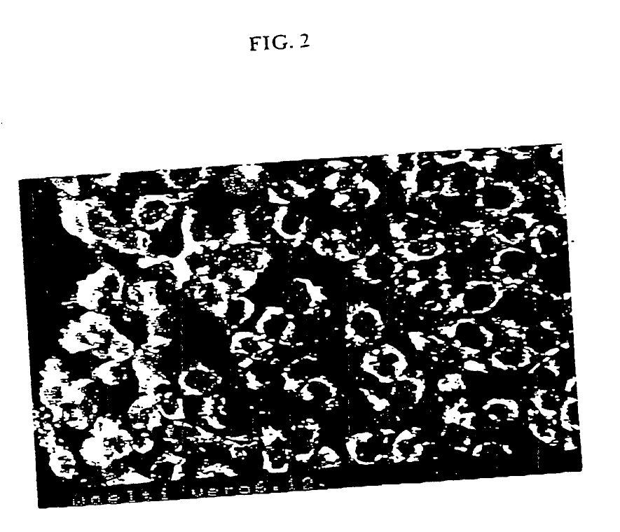

FIG. 2: Visualization of FITC plasmids through virosomes into Vero cells

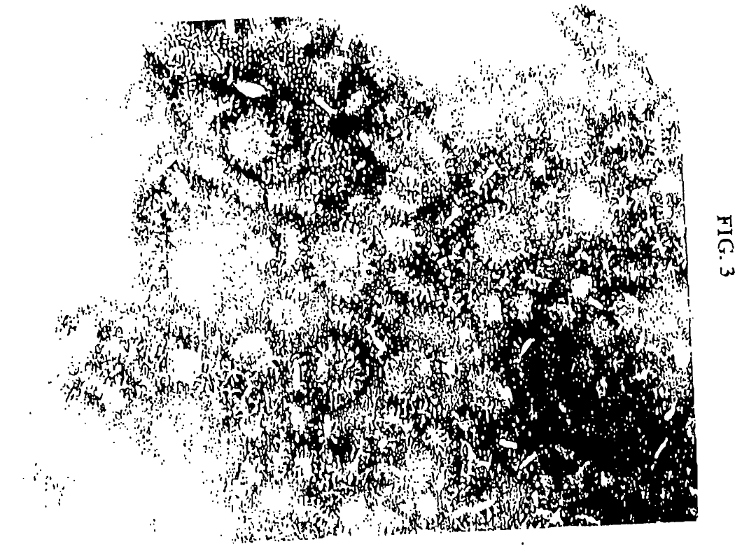

FIG. 3: Influenza virosomes containing plasmids expressing mumps F-antigen; negatively stained with phosphatungstate, magnification×100,000

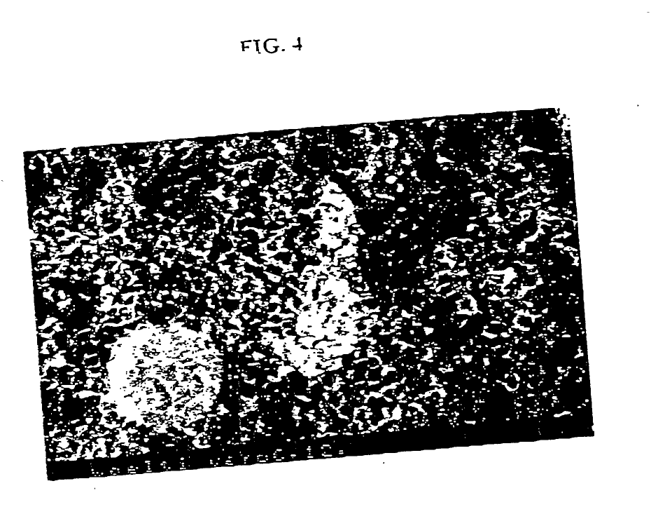

FIG. 4: pH fusion reaction of DOTAP-virosomes expressed as fluorescence dequenching

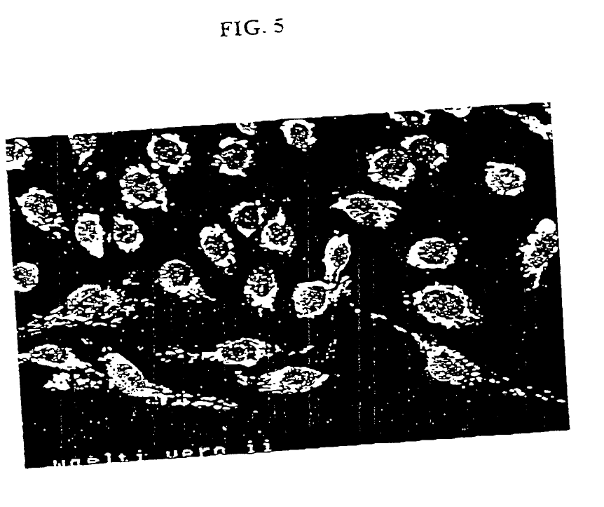

FIG. 5: Visualization of FITC Mumps plasmids through virosomes into Vero cells

The examples illustrate the invention.

### EXAMPLE 1

Construction of the Eukaryotic Plasmid Vector Expressing the HN and F Genes of Mumps Virus

The recombinant plasmids of the present invention can be produced by recombinant DNA techniques, such as those set forth generally by Maniatis et al., MOLECULAR CLONING, A Laboratory Manual, Cold Spring Harbor Laboratory (1982).

The hemagglutinin gene (1749 bp) of the Urabe strain of the mumps virus (Yamanishi et al., (1970), Studies on live mumps, vaccine ll. Evaluation of newly developed Live mumps vaccine. Biken Journal 13, 157-161) was amplified by reverse transcriptase-polymerase chain reaction (RT-PCR). RNA was extracted from viral genomic RNA, using the guanidinium thiocyanate-phenol-chloroform method, described by Chomczynski and Sacchi (1987, Anal. Biochem. 162). The synthesis of the cDNA was performed in a 25 μl reaction volume containing 50 mM KCl, 10 mM Tris-HCl pH 8.3, 5 mM MgCl2, 1 mM dNTP mixture (1 mM each), 20 U RNase inhibitor (Boehringer Mannheim, Germany) 40 U MMLV-RT (Perkin-Elmer Cetus, USA) and 0.75 mM of the sense primer after a denaturation step at 80° C. The mixture was incubated at 37° C. for 30 min, followed by 3 min denaturation at 94° C., and cooled on ice. The PCR was performed in a 100 μl volume containing 25 μl of the cDNA reaction, 10 μl of the PCR buffer (100 mM Tris-HCl pH 8.3, 500 mM KCl and 25 mM MgCl₂), 20 pmol each of sense primer (5′GGATCCAGATGGAGCCCTCGAAA3′) and antisense primer (5′GATCCTTATCAAGTGATAGTCMTCT3′), 0.125 mM dNTP mixture and 2 U of Taq polymerase (Perkin-Elmer Cetus, USA). The samples were subjected to 40 cycles of thermal cycling for 94° C. 1 min, 56° C. 40 s, 72° C. 90 s. Both the primers contained the restriction site for BamHI. The PCR product was purified with the QIAQUICK PCR purification kit (QIAGEN, Germany) and digested with the restriction enzyme BamHI (1.5 U) in a 100 μl volume containing the specific buffer (10 μl)(Boehringer Mannheim, Germany) at 37° C. overnight. The insert was then purified from the agarose gel by using the QIAquick gel extraction kit (QIAGEN, Germany) and cloned in plasmid pcDNA3 (InVitrogen) which had previously been cut by BamH1 and treated with the calf intestine phosphatase (CIP) (Boehringer Mannheim, Germany) in order to eliminate the circularization of the vector itself. Plasmid pcDNA3 is a 5.4 Kb vector containing the CMV promoter (bases 209-863), the BGH polyadenylation site (bases 1018-1249), the polylinker (bases 883-994), the SV40 promoter (bases 1790-2115) and the SV40 polyadenylation site bases 3120-3250). The recombinant plasmids, containing the HN gene of the Urabe strain (GC9) or the wild type (GC19) of the Mumps virus were used to transform the E. coli bacteria (DH5α strain) and some transformants were obtained. The DNA plasmids were recovered from the cells and the HN genes were sequenced by the dideoxy method using Sequenase (U.S. Biochemical Corp. Cleveland, Ohio, USA) as outlined in the protocol of the manufacturer. Large amount of the recombinant plasmid DNA was obtained in bacteria cells and purified with the Qiagen plasmid Kit (QIAGEN, Germany) in order to inject the DNA in mice. The concentration and purity of each DNA preparation was determined by OD260/280 readings. The 260/280 ratios were >1.8.

The same genes were inserted in another eukaryotic plasmid vector, pCMVβ (7.2 Kb) (Clontech, USA). This vector contains a CMV promoter, an RNA splice site, an SV40 polyadenylation site and the full length E. coli β-gal gene located within a pair of Not1 restriction sites (bases 820-4294) for excision and replacement with the HN gene of the Mumps virus (GD9 and GD19). Furthermore, the genes were inserted in another eukaryotic plasmid vector, pCI (4 Kb)(Promega, USA) which contains a CMV promoter, an SV40 polyadenylation site and a multiple cloning site where the HN gene of the Mumps virus was placed. The procedure followed for these new constructs was the same of the one above mentioned, except for the primers used for the amplification of the HN gene, both of which contained the Not1 restriction site (sense primer: 5′ GCGGCCGCAGATGGAGCCCTCGAAA3′ and anti-sense primer: 5′ GCGGCCGCTTATCAAGTGATAGTCAATCT3′).

### EXAMPLE 2

The F gene (1713 bp) of the Mumps virus (Urabe strain)(Cusi M. G. et al. Gene 161, 1995) deleted of the trans-membrane fragment (nt 1492) at the carboxy-terminal (GC 23) was amplified by RT-PCR from the virus genome and sequenced. The procedure used for this reaction was the same of the above mentioned. The primers used containing the BgI II site for the insertion in the pcDNA3 plasmid cut by BamHI were: sense primer 5′ACAGATCTGATCAGTMTCATGAA3′ and antisense primer 5′ACAGATCTTCAGGAGTTTACCTT3′. The primers used containing the Not1 site for the insertion in the pCMVβ (GD23) and pCI plasmid were sense primer 5′GCGGCCGCGATCAGTMTCATGM3′ and anti-sense primer 5′GCGGCCGCTCAGGAGTTTACCTT3′. The annealing temperature of these last primers was 60° C. in the PCR.

### EXAMPLE 3

The NP nucleocapsid (NP) gene (1657. nt) of the Mumps virus (Urabe strain) was amplified by RT-PCR from the virus genome. The procedure used for this reaction was the same of the above mentioned. The primers used containing the HindIII site for the insertion of the NP gene in the pcDNA3 vector (GC/NP) cut by HindIII were: sense primer 5′AAGCTTATGTCGTCTGTGCTCAAA3′ and anti-sense primer 5′AAGCTTCAGTGATTTACTCATCCC3′. The annealing temperature was 58° C. in the PCR.

### EXAMPLE 4

A chimera containing the Mumps virus F and HN genes linked by a linker was cloned in BamHI of the pcDNA3 vector. The F gene was deleted of the transmembrane fragment at the carboxy-terminal and the HN gene was deleted of its hydrophobic region at the amino terminal. The linker codes for 8 glycines and 2 serines; its sequence is: 5′GGTGGCGGTGGATCCGGTGGCGGCGGATCA3′.

### EXAMPLE 5

A new vector was obtained rom pcDNA3, after the deletion of a sequence coding for the resistance to the neomycin. pcDNA3 was cut by RsrII (at position 2796 nt) and SmaI (at position 2093 nt), treated with the Klenow polymerase and recircularized. It could be important not to vehiculate resistance to antibiotics in DNA vaccination or in gene therapy. The Mumps virus HN and F genes were also cloned in this vector (GC 42) as described above.

### EXAMPLE 6

From the pcDNA3 vector of the CMV promoter was deleted and a human desmin promoter was inserted upstream the multiple cloning site. The Mumps virus HN or F genes were cloned in BamHI and BgIII sites, respectively, as described above.

### EXAMPLE 7

The N gene (1176 bp) of the Respiratory Syncytial Virus was amplified by RT-PCR from the virus genome (wild type strain, isolated in the Siena Area, Italy), The synthesis of the cDNA was performed in a 25 μl reaction volume containing 50 mM KCl, 10 mM Tris-HCl pH 8.3, 5 mM MgCl₂, 1 mM dNTP mixture (1 mM each), U RNase inhibitor (Boehringer Mannheim Biochemicals, Germany) 40-U MMLV-RT (Perkin-Elmer Cetus, USA) and 0.75 mM of the sense primer (5′GCGGCCGCATGGCTCTTAGCAAAGTCAA3′) after a denaturation step at 80° C. The mixture was incubated at 37° C. for 30 min, followed by 3 min denaturation at 94° C. and cooled on ice. The PCR was performed in a 100 μl volume containing 25 μl of the cDNA reaction, 10 μl of the PCR buffer (100 mM Tris-HCl pH 8.3, 500 mM KCl and 2.5 mM MgCl₂), 20 pmol of sense primer (5′GCGGCCGCATGGCTCTTAGCAAAGTCM3′) and anti-sense primer (5′GCGGCCGCTCAAAGCTCTACATCA3′), 0.125 mM dNTP mixture and 2 U of Taq polymerase (Perkin-Elmer Cetus, USA). The samples were subjected to 40 cycles of thermal cycling for 94° C. 1 min, 60° C. 40 s, 72° C. 90 s. Both the primers contained the restriction site for Not I. The PCR product was purified with QIAQUICK PCR purification kit (QIAGEN, Germany) and digested with the restriction enzyme Not1 (1.5 U) in a 100 μl volume containing the specific buffer (10 μl)(Boeringer Mannheim, Germany) at 37 C overnight.

The insert was then purified from the agarose gel by using the Qiaquick gel extraction kit (QIAGEN, Germany) and cloned in pCMVβ and pCI previously cut by Not1 and treated with CIP.

### EXAMPLE 8

The S gene (875 bp) or the Pre-S1, Pre-S2, S ORF (1364 bp) of the Hepatitis B Virus was amplified by PCR from the plasmid containing the HBV genome (ATCC 45020). The synthesis of the DNA was performed in a 100 μl volume containing 200 ng of the DNA, 10 μl of the PCR buffer (100 mM Tris-HCl pH 5.3, 500 mM KCl and 25 mM MgCl2) 20 pmol of sense primer (5′GCGGCCGCATGGAGMCATCACATCA3′) for the S gene or sense primer (5′GCGGCCGCATGGGGCAGMTCTTTCCA3′) for the Pre-S1, Pre-S2, S ORF and antisense primer (5′GCGGCCGCTTAAATGTATACCCAAAGA3′), 0.125 mM dNTP′ mixture and 2 U of Taq polymerase (Perkin-Elmer Cetus, USA). The samples were subjected to 40 cycles of thermal cycling for 94° C. 1 min, 60° C. 40 s, 72° C. 90 s. Both the primers contained the restriction site for Not I. The PCR product was purified with the QIAQUICK PCR purification kit (QIAGEN, Germany) and digested with the restriction enzyme Not 1 (1.5 U in a 100 μl volume containing the specific buffer (10 μl)(Boehringer Mannheim, Germany) at 37° C. overnight. The insert was then purified from the agarose gel by using the QUIAquick gel extraction kit (QIAGEN, Germany) and cloned in pCMvp and pCT previously cut by Not1 and treated with CIP.

Demonstration that cells transfected with the recombinant constructs (GC9 and GC23) express the HN and the F proteins of the Mumps virus was done as follows:

Vero cells were grown at 37C, 5% CO2 in Dulbecco's modified Eagle's medium (DMEM) supplemented with 10% heat-inactivated foetal calf serum, and 100 mg/ml each of streptomycin and penicillin. Cells were seeded at 2×10⁵ cells/35 cm plate and, grown for 24 hr; after washing with phosphate buffered saline (PBS), they were transfected using Lipofectin (10 pg)(GIBCO, BRL) and plasmid DNA (2 pg)-Cells were harvested 72 hr after transfection and tested by immunofluorescence for the expression of the hemagglutinin. Cells were washed with PBS and fixed on slides with cold acetone. Monoclonal antibodies or rabbit polyclonal anti-serum (Swiss Serum Institute, Bern, Switzerland) were incubated with the transfected cells at 37° C. for 30 min. The cells were washed twice with PBS/2% foetal calf serum (FCS). Fluorescein isothiocyanate (FITC)-conjugated goat antibodies to mouse or rabbit Ig, diluted 1/100 in PBS/2% FCS, were added over 30 min at 37° C. The cells were washed twice in PBS and examined using a Diaplan microscope (J, eitz, Germany). Positive and negative controls were included in each test.

Demonstxation of the expression of Mumps virus hemagglutinin in mice immunized with recombinant DNA plasmid (GC9) was done as follows:

Four-week-old BALB/c female mice were obtained from Charles River Laboratories and were immunized two times at 4-week intervals in both hind legs with 50 pg of DNA (GC9) in 100 μl of saline. Ten animals were in each immunization group.

While under Ketamine-xylazine anesthesia, DNA (GC9 or pcDNA3) was administered intramuscularly. Ten days after the last immunization, mice were anesthetized and sacrificed. Serum, liver and muscle samples were collected from each mouse. Antibody responses were assayed by immunofluorescence (IF) test described by Just, M. Berger, R., Glucj, R., Wegmann, A. (1985): Feldvcrsuch mit ciner neuartigen human-diploiden Zellvakzine (HDCV) gegen Masem, Mumps und Riiteln Schweiz. Med. Wschr 115: 1727-I 730. DNA was extracted from muscle or liver sample (˜2 mg) of the immunized mice with phenol-chloroform, after proteinase K (200 pg/ml) digestion in a lysis buffer (25 mM EDTA, 75 mM NaCl, 0.01%SDS). The DNA collected after precipitation in cold ethanol (2.5 vol.) was submitted to the PCR assay. The PCR was performed in a final volume of 100 μl using 200 ng of DNA, Taq polymerase (2.5 U, Promega Corporation USA) in the specific buffer, with deoxyribonucleoside triphosphate mix (1.25 mM each) and 50 pmol of each primer (GIBCO, BRL). The primers used were located on the Mumps virus HN gene: sense primer 5′AAGGATCCATGGAGCCCTCGAAA3′ (nt 88-I 111) and the anti-sense primer 5′TAGGCATGTTGAGTGGATGG3′(nt 570-589). 40 cycles of PCR were performed (94° C. 1 min, 55° C. 50 s; 72 m C 1 min) in order to detect the presence of the recombinant plasmid in the immunized animals. To verify the suitability of the DNA samples for amplification, 300 ng of each sample DNA were tested for the amplification of the mouse globin gene, using specific primers (5′CACCTGACTGATGCTGAGM3′ and 5′ATTACCCATGATAGCAGAGG3′). All the samples were suitable for the amplification.

The presence of the recombinant DNA plasmid was revealed in the muscle sample of 3 out of 10 mice but in no one of the liver samples drawn ten days after the last immunization.

### EXAMPLE 9

Preparation of a Cationic Lipid Vesicle With Fully Fusion Active Viral Hemagglutinin Trimers from Influenza Virus Containing Encapsulated Mumps Plasmid Expressing the HN Gene “Naked” DNA.

Before “naked” DNA technology can prophylactically be applied to a vaccine in need thereof, a number of technical problems, particularly relating to the development of a suitable carrier system, need to be solved beforehand. For instance, genetic material such as e.g., a plasmid, can be unstable and break down or be otherwise more or less inactivated before it reaches the target cells and it may thus be necessary to use large quantities of such material. Due to these large amounts a question arises about the potential risk in the human or animal body. By using the cationic virosomes of the present invention as carriers for the plasmid these problems can be successfully overcome and potential toxicity can be considerably decreased. This beneficial effect is achieved because the present cationic virosomes have far higher activity and efficiency for the transfer of entrapped material, particularly of genetic material such as plasmids expressing mumps genes, into target cells than liposomes or normal virosomes known hitherto.

Preparation of DOTAP Virosomes

Hemagglutinin (HA) from the A/Singapore/6/86 Strain of influenza virus was isolated as described by Skehel and Schild (1971), Proc. Natl. Acad. Sci. USA 79: 968-972. In short, virus was grown in the allantoic cavity of hen eggs, and was purified twice by ultracentrifugation in a sucrose gradient. Purified virus was stabilized in a buffer containing 7.9 mg/ml NaCl, 4.4 mg/ml trisodiumcitrate −2H₂O, 2.1 mg/ml 2-morpholinoethane sulfonic acid, and 1.2 mg/ml N-hydroxyethyl-piperazine-N′-2-ethane sulfonic acid, pH 7.3. 53 ml of the virus suspension containing 345 pg HA per ml were pelletted by ultracentrifugation at 100,000×g for 10 min. 7.7 ml of a buffered detergent solution containing 145 mM NaCl, 2.5 mM HEPES and 54 mg/ml of the non-ionic detergent octaethyleneglycol monododecylether (OEG=C₁₂E₈), pH 7.4 were added to the influenza virus pellet. The pellet was completely dissolved by using ultrasonication for 2 min at room temperature. The solution was subjected to ultracentrifugation at 100,000×g for 1 hour. The obtained supernatant contained the solubilized HA trimer (1.635 mg HA/ml) and trace amounts of neuramidase. 6 mg of DOTAP were added to 3.7 ml of supernatant (6 mg HA) and dissolved. The solution was sterilized by passage through a 0.2 um filter and then transferred to a glass container containing 1.15 g of sterile Biobeads SM-2. The container was shaken for 1 hour by using a shaker REAX2 from Heidolph (Kelheim, Germany). This procedure was repeated three times with 0.58 mg of Biobeads. After these procedures a slightly transparent solution of DOTAP virosomes was obtained.

### EXAMPLE 10

Preparation of DOTAP-Phosphatidylcholine (PC)-Virosomes.

HA was isolated according to Example 9. To the supernatant containing the solubilized HA trimer (6 mg HA), 5.4 mg DOTAP and 0.6 mg PC were added and dissolved. The formation of virosomes was obtained according to Example 9.

### EXAMPLE 11

Preparation of DOTAP-PC-PE-Virosomes

HA was isolated according to Example 9. To the supernatant containing the solubilized HA trimer (6 mg HA), 2.7 mg DOTAP, 0.6 mg PC and 2.7 mg PE were added and dissolved. The formation of virosomes was obtained according to Example 9.

### EXAMPLE 12

Incorporation of Plasmids Expressing Mumps Genes into DOTAP Virosomes.

The plasmids of Example (1) were used for the demonstration of the high efficiency of cationic virosomes in transfection. 5′-FITC plasmids were synthesized via phosphoramidite chemistry (Microsyth GmbH, Balgach, Switzerland). A mixed sequence control (msc) plasmid consisting of the same length of nucleotides as the FITC-plasmid was used.

1 ml of DOTAP virosomes or DOTAP-PC virosomes was added to each of

a)2 mg of FITC-plasmid (1.3 pmol), and

b)_{3.1} mg plasmid (1.3 pmol)

The FITC-plasmids and plasmids were incorporated into DOTAP virosomes according to Example 9. Non-encapsulated plasmids were separated from the virosomes by gel filtration on a High Load Superdex 200 column (‘Pharmacia, Sweden). The column was equilibrated with sterile PBS. The void volume fraction containing the DOTAP virosomes with encapsulated plasmids were eluted with PBS and collected. Virosome-entrapped FITC plasmid concentrations were determined fluorometrically after the virosomes were fully dissolved in 0.1 M NaOH containing 0.1%(v/v) Triton X-100. For calibration of the fluorescence scale the fluorescence of empty DOTAP-virosomes that were dissolved in the above detergent solution was set to zero.

DOTAP-virosomes with encapsulated plasmids were used for transfection experiments in vitro and in vivo.

FIG. 1 shows the mumps antigen expression of Vero cells which were incubated four days before with. DOTAP virosomes encapsulating mumps plasmids. The mumps antigen expression is expressed through staining with a fluorescent polyclonal antibody from rabbit against mumps virus.

DOTAP-virosomes with encapsulated FITC plasmids were used for visualization of the high transfer-rate of plasmid through virosomes into Vero cells (FIG. 2). No fluorescence could be detected after giving the same amount of FITC-plasmid without virosomal encapsulation.

### EXAMPLE 13

Electron Microscopy Observations.

Micrographs of DOTAP virosomes confirm the unilamellar structure of the vesicles with an average diameter of approximately 120 to 180 nm as determined by laser light scattering. The HA protein spikes of the influenza virus are clearly visible (FIG. 3).

### EXAMPLE 14

Determination of the Fusion Activity of DOTAP Virosomes.

The fusion activity of the present DOTAP virosomes was measured by the quantitative assay based on fluorescence dequenching described by Hoekstra et al. (1984), Biochemistry 23: 5675-5681 and Luscher et al. (1993), Arch. Viral. 130: 317-326. The fluorescent probe octadecyl rhodamine B chloride (RI8) (obtained from Molecular Probes Inc., Eugene, USA) was inserted at high densities into the membrane of DOTAP virosomes by adding the buffered OEG (C₁₂E₈) solution containing DOTAP and HA to a thin dry film of the fluorescent probe, followed by shaking for 5 to 10 min for dissolving the probe, then continuing as described above under “Preparation of a cationic vesicle”. Dilution of the quenching rhodamine was observed by incubation of the rhodamine-labeled DOTAP virosomes with model liposomes (ratio of DOTAP:liposomal phospholipid=1:20). The fluorescence was measured by a Perkin-Elmer 1000 spectrofluorimeter at 560 and 590 nm excitation and emission wavelengths, respectively. FIG. 4 shows the pH-induced fusion reaction of DOTAP virosomes expressed as percent of fluorescence dequenching (% FDQ).

### EXAMPLE 15

Time of Cellular Uptake of Virosome Encapsulated GC/9 Plasmid-FITC.

It proved very useful to label the plasmid with fluorescein to study the mechanism of cellular uptake of DOTAP virosomes.

Vero cells were grown in 2-well tissue culture chamber slides (Nunc, Naperville, Ill. 60566, USA). 50 μl of FITC-mumps plasmid virosomes were added to the cells. They were incubated for 5, 15, and 30 min at 37 C, washed twice with PBS and then examined by fluorescence microscopy. DOTAP virosomes with encapsulated FITC-mumps-plasmid were rapidly incorporated into the cells as can be seen in FIG. 5.

Examination of the biological effect of mumps plasmid-FITC-DOTAP virosomes measured by the chymidine incorporation method.

Vero cells were cultured in 24-well Costar plates at an initial concentration of 1×10⁵ per well and per ml. After an incubation of 24 hours, medium was removed and 625 μl of fresh medium containing 0.5 μCi ¹⁴C-thymidine (prepared from [2-¹⁴C]thymidine, 52.0 mCi/mmol; Amersham, England) and 75 μl of DOTAP virosomes containing 0.2 nmol of either mumps plasmid or FITC-mumps plasmid were added. The cultures were gently shaken at very slow agitation for 1 hr at 37° C. and then transferred to the incubator. After 48 hours the cell suspension was removed, transferred to centrifuge vials, and centrifuged. Obtained cell pellets were washed twice. When the cells could not sufficiently be dispersed into a single cell suspension, they were exposed briefly to a trypsin/EDTA solution.

Cell pellets were dissolved in 1.5 ml of a 0.1 M NaOH/Triton-X-100 (0.1%) solution. 3 ml of liquid scintillation cocktail (Ready Protein -!-, Beckman, Fullerton, Calif., USA) were added to 1 ml of solution. ¹⁴C-radioactivity was counted in a liquid scintillation counter (Beckman, Full&on, CA, USA).

This experiment showed the extraordinary uptake and transfection efficiency of mumps plasmid virosomes: Almost trace amounts of 75 pMol/well and per ml of virosomal mumps plasmid in the cells are detectable with this method.

### EXAMPLE 16

Alternative Preparation of a Cationic Lipid Vesicle With, Fully Fusion Active Viral Hemagglutinin Trimers from Influenza Virus Containing the Encapsulated Mumps Plasmid.

Preparation of DOTAP Virosomes and Incorporation of Mumps Plasmid Expressing the HN Antigen.

4 mg of DOTAP were dissolved in 0.5 ml of the buffered detergent solution containing 145 mM NaCl, 2.5 mM HEPES and 54 mg/ml of OEG (C₁₂E₈), pH 7.4. To the resulting mixture 100 pg of mumps plasmid were added and dissolved. The solution was subjected to ultrasonication for 30 seconds. OEG was removed by Biobeads as described in Example 9. A second mixture of NaCl, HEPES and OEG, 3 mg PC, 1 mg PE and 1 mg HA were subjected to the same biobeads treatment to form neutral virosomes. The DOTAP plasmid liposomes were fused with the neutral HA-virosomes by treatment with ultrasonication during 60 seconds.

Transfection of DOTAP Virosomes Loaded With Mumps Plasmid into Vero Cells.

The obtained solution was diluted 1:1000 with PBS. 20 μl and 50 μl of this solution containing 1 ng and 2.5 ng plasmid, respectively, were added to 2×10⁶ Vero cells. After 48 h incubation the supernatants of the cell cultures were tested for HN antigen by an ELISA assay. A content of 20 to 45 pg HN per ml was measured.

Comparison of Transfection Efficiency of Mumps Plasmid (HN) Loaded DOTAP Virosomes With Mumps Plasmid Loaded DOTAP Liposomes.

No HN was found in myeloma cell cultures transfected with DOTAP liposomes (i.e., devoid of viral fusion peptides on the membrane) containing the same amount of plasmid as the DOTAP virosomes. In order to obtain the same transfection results as with the plasmid loaded DOTAP virosomes it was necessary to increase the amount of plasmid DNA loaded DOTAP liposomes by a factor of one thousand (1000).

### EXAMPLE 17

Humoral and Cellular Immune Response to Viral Mumps-Antigens Induced by Genetic Immunization

BALB/c mice (5 animals per group) were injected intramuscularly with “naked” plasmid DNA or with virosomal plasmid DNA. The response was read out 4 weeks postimmunization. Mean values (2 SD) are given.

Cytotoxicity Assay for Specific T Cell Reactivity, (Refers to 1 in the Table 1)

Spleen cells from immunized mice were suspended in a-MEM tissue culture medium supplemented with 10 mM HEPES buffer, 5×10⁻⁵ M 2-β mercaptoethanol, antibiotics and 10% VN fetal calf serum. 3×10⁷ responder cells were cocultured with 1.5×10⁶ syngeneic, mumps-antigen (HN)-expressing or mumps-antigen (F)-expressing transfectants (irradiated with 20'000 rad) in 10 ml medium in upright 25 cm² tissue culture flasks in a humidified atmosphere/7% CO2 at 37° C. Cytotoxic effector populations were harvested after varying intervals of in vitro culture and washed twice. Serial dilutions of effector cells were cultured with 2×10^{3 51}Cr-labeled targets in 200 μl round-bottom wells. Specific cytolytic activity of cells was tested in short-term ⁵¹Cr-release assays against transfected (As+) or non-transfected (Ag−) control targets. After a 4 h incubation at 37 C, 100 μl of supernatant were collected for g-radiation counting. The percentage specific release was calculated as [(experimental release−spontaneous release)/(total release−spontaneous release)]×100. Total counts were measured by resuspending target cells. Spontaneously released counts were always less than 20% of the total counts. Data shown represent mean specific lysis values of 5 mice (5 SD) (Tab. I).

Determination of Specific Serum Antibody Levels. (Refers to 2 in the Table 1)

Antibodies against mumps virus were detected in mouse sera using an immune fluorescence test described by Just, M. Berger. R., Gluck, R., Wegmann. A. (1985) Feldversuch mit einer neuarrigen human-diploiden Zellvakine (HDCV gegen Masern. Mumps und Rotein. Schweiz Med Wschr 115: 1727-I 730. Concentrations of anti-mumps were standardized against a WHO-reference standard.

The tested sera were diluted so that the measured OD values were between standard serum one and six. Values presented in this paper are calculated by multiplying the serum dilution with the measured antibody level (mIU/ml). Serum titers shown are the mean of 5 individual mice (±SD)(Tab. 1).

TABLE 1

| Mice immunized | μg DNA/ | Cytotoxic T response¹ | | Ilamoral All |
|---|---|---|---|---|
| with plasmid | mouse | Ag⁺ target | Ag⁻ target | response² log 2 |
| GC/9 | 25 | 34 ± 11 | 4 ± 3 | 3.2 ± 1.2 |
| GC/9 | 5 | 11 ± 8 | 8 ± 5 | 1.7 ± 1.1 |
| GC/9 | 1 | 7 ± 3 | 4 ± 2 | 1.4 ± 0.8 |
| GC/23 | 25 | 43 ± 12 | 4 ± 1 | 7.9 ± 1.6 |
| GC/23 | 5 | 19 ± 10 | 5 ± 3 | 1.7 ± 0.8 |
| GC/23 | 1 | 11 ± 3 | 6 ± 4 | 0.9 ± 0.4 |
| GC/NP | 25 | 65 ± 19 | 7 ± 5 | 1.6 ± 0.3 |
| GC/NP | 5 | 30 ± 21 | 5 ± 3 | 1.3 ± 0.7 |
| GC/NP | 1 | 9 ± 5 | 7 ± 4 | 1.4 ± 0.6 |
| Virosomal GC/9 | 25 | 89 ± 14 | 8 ± 5 | 4.7 ± 3.7 |
| Virosomal GC/9 | 5 | 81 ± 21 | 9 ± 6 | 4.2 ± 1.9 |
| Virosomal GC/9 | 1 | 74 ± 16 | 7 ± 1 | 4.3 ± 2.1 |
| Virosomal GC/23 | 25 | 131 ± 29 | 4 ± 4 | 5.6 ± 3.5 |
| Virosomal GC/23 | 5 | 147 ± 19 | 6 ± 3 | 5.9 ± 2.4 |
| Virosomal GC/23 | 1 | 98 ± 21 | 7 ± 3 | 5.8 ± 2.6 |
| Virosomal GC/NP | 25 | 181 ± 51 | 6 ± 4 | 3.8 ± 0.6 |
| Virosomal GC/NP | 5 | 170 ± 43 | 9 ± 2 | 2.6 ± 0.7 |
| Virosomal GC/NP | 1 | 172 ± 44 | 3 ± 1 | 2.3 ± 0.9 |

Description of Table 1:

The data show that all plasmids induced humoral and CTL-immune response in mice. However, it was evident that the virosomal constructs showed a significantly higher effect than the “naked” DNA plasmids. As expected, the GC/NP-plasmid yielded a very low humoral immune response, be it as “naked” or as “virosomal” preparation; in contrast to the low antibody induction, the cellular immune response was high, again especially with the virosomal preparation.

### EXAMPLE 18

Challenge Experiments in the Newborn Hamster Model

The protective capacity of virosomal mumps plasmid (GC9 and GC23) was evaluated in a conventional newborn hamster model as described previously, by e.g. Overman et al., 1953; Burr and Nagler, 1953; Love et al., Microb. Pathog. 1 (1986). 149-158; J. Virol. 58 (1986), 220-222; Develop. Neurosc. 7 (1985), 65-72; J. Virol. 53 (1985), 67-74 and references cited therein.

The experiments proceeded in several steps:

(A) Female hamsters were vaccinated i. m with each of the above virosomal plasmid constructs and with a control empty virosomal preparation (5 pg/animal).

(B) Antibody titers towards the specific proteins were measured periodically for several weeks after vaccination to ensure that an immune response was generated.

(C) Immune female hamsters were then mated to obtain newborn animals for the actual challenge experiment.

(D) Newborn hamsters were inoculated intracerebrally with the Kilham strain of Mumps virus (9×10⁵ pfu per animal) and mortality due to encephalitis was followed for 10 days after challenge.

TABLE 2
RESULTS

| | Mumps | |
|---|---|---|
| | Ab titer | Overall survival |
| Virosomal plasmid | in mothers | in offspring after in |
| used for immunization | log2 | challenge (10 days p. inf.) |
| Empty virosomes | <1.2 | 33.3% (5 surv./15 challenged) |
| (control) | | |
| Virosomal GC/23 (F protein) | 6.7 | 44.4% (4 surv./9 challenged) |
| Virosomal GC/9 (HN protein) | 6.1 | 100% (4 surv./4 challenged) |
| Virosomal GC/DC (F + HN) | 7.5 | 100% (5 surv./5 challenged) |

The mortality rate for newborn hamsters originating from hamster mothers immunized with virosomal GC23 (F) or GC9 (HN) was reduced, indicating that the anti F and anti HN antibodies passed from the mothers to their offspring have the capacity to counteract the infection by the Mumps virus. The best result was obtained with the dicistronic-virosomal construct.

### EXAMPLE 19

Humoral and Cellular Immune Response to Viral Mumps-Antigens Induced by Intranasal Genetic Immunization in Mice

Female BALB/c mice 4 weeks old (Charles River) were used. Mice were anesthetized with ketamine-xylazme and immunized in. with 30 μl (less then 1 μg of DNA) of virosomes-DNA or virosomes alone. The mice inhaled these preparations simply by breathing. The same procedure was used for repeated immunizations one, three, and four weeks after the first inoculation. Group A, B and C were immunized with the plasmid expressing the mumps virus HN protein (GC9), groups D, E and F received the plasmid coding the mumps virus F antigen (GC23). Groups A and D received an intramuscular priming with influenza virus vaccine (100 μl containing 3 pg of HA).

Group G received the vector plasmid pcDNA3 entrapped into virosomes. Each group was represented by 5 mice. For collection of bronchoalveolar lavages (BAL) and nasal washes (NW) mice were sacrificed by cervical dislocation under anesthetization. Collection of bronchoalveolar lavages (BAL) and nasal washes (NW) from mice were performed as described elsewhere (Takao S-I, Kiyotani K., Sakaguchi T., Fujli Y., Seno M., Yoshida T. 1997-Protection of mice from respiratory Sendai virus infections by recombinant vaccinia viruses. J Virol. 71: 832-838.).

Elisa

Mumps virus-specific IgG and IgA antibodies were measured by enzyme-linked immunosorbant assay (ELISA). Purified virions of mumps virus were diluted in coating buffer (0.05 M NaHCO₃/Na2CO3, pH 9.6) to 1 μg of protein per ml, and dispensed to a 96 well plate at 100 μl/well. After allowing to absorb overnight at 4 C, the wells were washed with PBS-0.05% Brij 35 and blocked for preventing nonspecific binding by incubation with 5% heat inactivated foetal calf serum (FCS) in PBS-Brij 35 for 2 h at room temperature.

A 100 μl aliquot of samples were twofold diluted in the plate and allowed to react for 1 h at 37° C. The plate was then washed, and 100 μl of goat horseradish peroxidase-labelled anti-mouse IGg (γ)antiserum (I/8000)(BioRad, Milan, Italy) for IgG ELISA or goat anti-mouse IgA (α)antiserum (I/6000)(Southern Biotechnology Associates, Inc., USA) for IgA ELISA was added and the plate was incubated for 1 h at 37C. After washing, 3,3′,5,5′ Tetramethylbenzidine (TMB) (Sigma, Milan, Italy) was added and allowed to react at room temperature for 30 min, and the reaction was stopped with 100 μl of 1 NH₂SO₄. Colorimetric conversion for the substrate was measured in a microplate spectrophotometer at 4.50 nm (Behring, Milan, Italy), Titers of samples were calculated from endpoint dilutions showing an optical density of more than 0.2 above the background represented by the negative control serum. The results of the Elisa are summarized in Table 3 below.

| Mice | Serum IgG | BAL IgA | NW IgA |
|---|---|---|---|
| Group A | 356 ± 115 | B ± 7 | 10 ± 5 |
| Group B | 15 ± 20 | 11 ± 6 | 10 ± 4 |
| Group C | 12 ± 7 | neg | neg |
| Group D | 157 ± 160 | 4 ± 8 | 13 ± 3 |
| Group E | 16 ± 9 | 5 ± 7 | 8 ± 3 |
| Group F | 19 ± 22 | neg | neg |
| Group G | neg | neg | neg |

Values correspond to the Ab-geometric mean titer (GMT)f Standard Deviation (SD)

As shown in Table 3, considering the ratio between the total level of IgG and the virus specific IgG1 or IgG2a, the amount of IgG2a isotype was predominant in group A immunized with GC9-virosomes, whereas the amount of IgG1 isotype was predominant in group D immunized with GC23-virosomes, indicating Th2 a response.

Cytolcine Assays

Splenocytes were cultured as described above with the same panel of antigens, except that after 24 h in culture, cell-free supernatants were harvested for the presence of IL-2 and after 48 h for the presence of IFN-γ IL-4 and IL-10. Samples were stored at −80C. Briefly, microtiter plates were coated overnight at 4C with 100 ul of anti-cytokine capture Mab (Pharmingen, Milan, Italy) at 1 ug/ml. The plates were washed twice with PBS-Tween and blocked with 100 μl of 10% FCS in PBS per well per 2 h at room temperature. Then the plates were washed twice and incubated with duplicates of serially diluted samples and standards (Sigma) overnight at 4° C. Then 100 μl of the biotinylated anticytokines MAb at 1 μg/ml was added to each well and the mixture was incubated at room temperature for 1 h. The plates were then washed three times, 100 μl of streptavidin-peroxidase (1/1000)(Sigma) was added, and the mixture was incubated at room temperature for 30 min. Following multiple final washings, the color was developed with TMB (Sigma) and stopped with 100 μl of 1 N H2SO4 and the absorbance at 405 nm was measured with an ELISA plate reader. The concentration of cytokines in samples was determined from the standard curve.

All the experiments described thereafter were performed using splenic cells taken twelve days after immunization. Table 4 summarizes representative measurements obtained from two separate experiments.

| Group | IL-2 | IFN-γ | IL-4 | IL-10 |
|---|---|---|---|---|
| A | 300 | 300 | 150 | 0 |
| B | 150 | 625 | 0 | 0 |
| C | 300 | 100 | 0 | 0 |
| D | 150 | 100 | 0 | 0 |
| E | 600 | 100 | 0 | 0 |
| F | 0 | 675 | 0 | 0 |
| G | 0 | 0 | 0 | 0 |

Values are given in pg/ml.

Mumps virus-stimulated cells from mice inoculated with DNA-virosomes induced the production of IL-2 and IFN-γ, whereas it induced the production of IL-2 and IL-10 in cells taken from mumps virus-immunized animals. Immunization with DNA-virosomes such as the control immunization with the purified antigens correlated with ThI phenotype.

Abbreviations used in the description:

CAT chloramphenicol acetyltransferase

DOTAP N-[(1,2,3-dioleoyloxy)-propyl]-N, N,N-timethylammonium-methylsulfate

DOTMA N-[(1,2,3-dioleoyloxy)-propyl]-N, N, N-trimethylammonium-chloride

FITC-OPT fluorescein isothiocyanate-labeled oligodeoxyribonucleotide phosphorothioate

HA hemagglutinin

OEG (C12E8)octaethyleneglycol monododecylether

PC phosphatidylcholine

PE phosphatidylethanolamine

PNA peptide nucleic acid

MPB. PE N-[4-(p-maleimido-)-phenylbutyryl]-phosphatidylethanolamine

neg Negative

1. A vaccine comprising a virosome, said virosome comprising
a) a cationic lipid;
b) an influenza hemagglutinin protein (HA) or a derivative thereof which is biologically active and capable of inducing the fusion of said virosome with cellular membranes and of inducing the lysis of said virosome after endocytosis by antigen presenting cells, and
c) a nucleic acid comprising a nucleic acid encoding an antigen derived from a pathogen located in the inside.

2. The vaccine according to claim 1, wherein said cationic lipid is an organic molecule that contains a (poly)cationic component and a nonpolar tail, wherein said (poly)cationic component comprises at least one member selected from the group consisting of:
N-[1,2,3-dioleoyloxy)propyl]-N, N, N-trimethylammonium chloride (DOTMA)
N-[1,2,3-dioleoyloxy)propyl]-N,N, N-trimethylammoniummethylsulfate (DOTAP)
N-t-butyl-N′-tetradecyl-3-tetradecylaminopropionamidine; and
the polycationic lipids comprise at least one member selected from the group consisting of
1,3-dipalmitoyl-2-phosphatidylethanolamido-spermine (DPPES), dioctadecylamidoglycyl spermine (DOGS),
2,3-dioleyloxy-N-[sperminecarboxamido)ethyl]-N, N-dimethyl-1-propane aminiumtrifluoro-acetate (DOSPA),
1,3-dioleoyloxy-2-(6-carboxy-spermyl)-propylamide (DOSPER) and
N,N,N′,N′-tetramethyl-N,N′-bis(2-hydroxyethyl)-2,3-dioleoyloxy-1,4-butanediammonium iodide (THDOB).

3. The vaccine according to claim 1, wherein said nucleic acid is DNA.

4. The vaccine according to claim 1, wherein said nucleic acid is RNA.

5. The vaccine according to claim 1, wherein said nucleic acid is a polycistronic nucleic acid.

6. The vaccine according to claim 5, wherein said polycistronic nucleic acid comprises a suicide gene that is preferably inducible with a therapeutically acceptable drug.

7. The vaccine according to claim 1, wherein said pathogen is a bacterium, a prion, a parasite or a virus.

8. The vaccine according to claim 7, wherein said virus is a single-stranded, non-segmented genome negative-sense RNA virus, preferably of the family Paramyxoviridae and most preferably mumps virus or measles virus.

9. The vaccine according to claim 1, wherein said nucleic acid is a recombinant vector.

10. The vaccine according to claim 9, wherein said recombinant vector contains the hemagglutinin-neuraminidase antigen of mumps virus, the fusion protein of mumps virus and the nucleoprotein of mumps virus.

11. The vaccine according to claim 1, where said HA derivate is the HA fusion peptide.

12. A vaccine comprising a vector encoding the hemagglutinin-neuraminidase antigen of mumps virus, the fusion protein of mumps virus and the nucleoprotein of mumps virus.

13. The vaccine according to claim 10 or 12, wherein said vector is GC9, GC23, GCNP or GCDC.

14. A method of stimulating the immune system of a patient in need thereof, comprising administering a suitable dosage of the vaccine according to claim 1 or 12.

15. A method for the prophylaxis of infectious diseases comprising administering a suitable dosage of the vaccine according to claim 1 or 12 to a patient in need thereof.

16. The vaccine of claim 1 or 12, wherein said vaccine is designed to be administered via nasal routes.

17. The method of claim 14, wherein said vaccine is designed to be administered via nasal routes.

18. The method of claim 15, wherein said vaccine is designed to be administered via nasal routes.
